(19) United States
(12) Patent Application Publication
(10) Pub. No.: US 2014/0318546 A1
(43) Pub. Date: Oct. 30, 2014

(54) RESPIRATORY MASK WITH CONDENSED LIQUID OUTLET

(71) Applicant: KONINKLIJKE PHILIPS N.V., EINDHOVEN (NL)

(72) Inventor: Richard Thomas Haibach, Pittsburgh, PA (US)

(21) Appl. No.: 14/345,664

(22) PCT Filed: Sep. 26, 2012
(86) PCT No.: PCT/IB2012/055113

§ 371 Date: Mar. 19, 2014

Related U.S. Application Data
(60) Provisional application No. 61/539,514, filed on Sep. 27, 2011.

(51) Int. Cl. A61M 16/08; A61M 16/00; A61M 16/06

(52) U.S. Cl. CPC A61M 16/0808; A61M 16/0622; A61M 16/0057
USPC 128/205.25; 128/206.24

(57) ABSTRACT

A patient interface device (8) includes a patient sealing assembly (12) structured to engage a face of the patient when the patient interface device is donned by a patient. The patient sealing assembly has a bottom region (36), a top region (38) opposite the bottom region, and a side region (40, 42) extending from the bottom region to the top region, wherein one or more orifices (48a, 48b) are provided in the side region, the one or more orifices each extending from an interior of the patient sealing assembly to an exterior of the patient sealing assembly and being structured to allow a liquid that condenses within the patient sealing assembly to drain from the patient sealing assembly.

This patent application claims the priority benefit under 35 U.S.C. §119(e) of U.S. Provisional Application No. 61/539,514 filed on Sep. 27, 2011, the contents of which are herein incorporated by reference.

### BACKGROUND OF THE INVENTION

1. Field of the Invention

The present invention relates to respiratory therapy systems, such as non-invasive ventilation and pressure support systems, and, in particular, to a patient interface device for a respiratory therapy system that includes a mechanism for reducing the adverse effects of rainout during therapy.

2. Description of the Related Art

There are numerous situations where it is necessary or desirable to deliver a flow of breathing gas non-invasively to the airway of a patient, i.e., without intubating the patient or surgically inserting a tracheal tube in their esophagus. For example, it is known to ventilate a patient using a technique known as non-invasive ventilation. It is also known to deliver positive airway pressure (PAP) therapy to treat certain medical disorders, the most notable of which is obstructive sleep apnea (OSA). Known PAP therapies include continuous positive airway pressure (CPAP), wherein a constant positive pressure is provided to the airway of the patient in order to splint open the patient's airway, and variable airway pressure, wherein the pressure provided to the airway of the patient is varied with the patient's respiratory cycle. Such therapies are typically provided to the patient at night while the patient is sleeping.

Non-invasive ventilation and pressure support therapies as just described involve the placement of a patient interface device including a mask component having a soft, flexible cushion on the face of a patient. The mask component may be, without limitation, a nasal mask that covers the patient's nose, a nasal cushion having nasal prongs that are received within the patient's nares, a nasal/oral mask that covers the nose and mouth of the patient, or a full face mask that covers the patient's face. Such patient interface devices may also employ other patient contacting components, such as forehead supports, cheek pads and chin pads. The patient interface device is connected to a gas delivery tube or conduit and interfaces the ventilator or pressure support device with the airway of the patient, so that a flow of breathing gas can be delivered from the pressure/flow generating device to the airway of the patient. It is known to maintain such devices on the face of a wearer by a headgear having one or more straps adapted to fit over/around the patient's head.

Rainout is a phenomenon wherein liquid (e.g., water) condenses and puddles within the mask component of a patient interface device during therapy. Rainout occurs as warm humidified air from the ventilator or pressure support device passes through the lower temperature gas delivery tube or conduit and mask component on its way to the patient, causing it to condense. For the patient, this can be a problem, as they may wake up with a bothersome puddle in their patient interface device. In order to reduce the potential for rainout, it is known to use a heated delivery tube and/or mask component to reduce the likelihood of condensation. Such a set-up, however, is costly and requires additional current to operate. Patient circuit insulators have also been used to reduce the potential for rainout, but such configurations tend to be bulky and thus not comfortable for the patient.

### SUMMARY OF THE INVENTION

Accordingly, it is an object of the present invention to provide a patient interface device that overcomes the shortcomings of conventional patient interface devices. This object is achieved according to one embodiment of the present invention by providing a patient interface device that includes a mechanism for reducing the adverse effects of rainout during therapy.

It is yet another object of the present invention to provide a method of providing respiratory therapy that does not suffer from the disadvantages associated with conventional respiratory therapy techniques. This object is achieved by providing a method that alleviates the effects of rainout by draining condensed liquids from the patient interface device.

In one embodiment, a patient interface device is provided that includes a patient sealing assembly structured to engage a face of the patient when the patient interface device is donned by a patient. The patient sealing assembly has a bottom region, a top region opposite the bottom region, and a side region extending from the bottom region to the top region, wherein one or more orifices are provided in the side region, the one or more orifices each extending from an interior of the patient sealing assembly to an exterior of the patient sealing assembly and being structured to allow a liquid that condenses within the patient sealing assembly to drain from the patient sealing assembly.

In another embodiment, a method of providing respiratory therapy to a patient is provided that includes providing a flow of breathing gas to a patient through a patient interface device, the patient interface device including a patient sealing assembly engaging a face of the patient, the patient sealing assembly having a bottom region, a top region opposite the bottom region, and a side region extending from the bottom region to the top region, wherein one or more orifices are provided in the side region, the one or more orifices each extending from an interior of the patient sealing assembly to an exterior of the patient sealing assembly, and draining a liquid that condenses within the patient sealing assembly during the therapy from the patient sealing assembly through the one or more orifices.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is a schematic diagram of a system adapted to provide a regimen of respiratory therapy to a patient according to one exemplary embodiment;

FIG. 2 is a top plan view and FIG. 3 is a side elevational view of a patient interface device forming part of the system of FIG. 1 illustrating a first predefined area in which drainage orifices may be provided;

FIG. 4 is a front elevational view and FIG. 5 is a side elevational view of a patient interface device forming part of the system of FIG. 1 illustrating a first predefined area in which drainage orifices may be provided; and

FIGS. 6-10 are schematic diagrams of systems adapted to provide a regimen of respiratory therapy to a patient according to various alternative exemplary embodiments.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

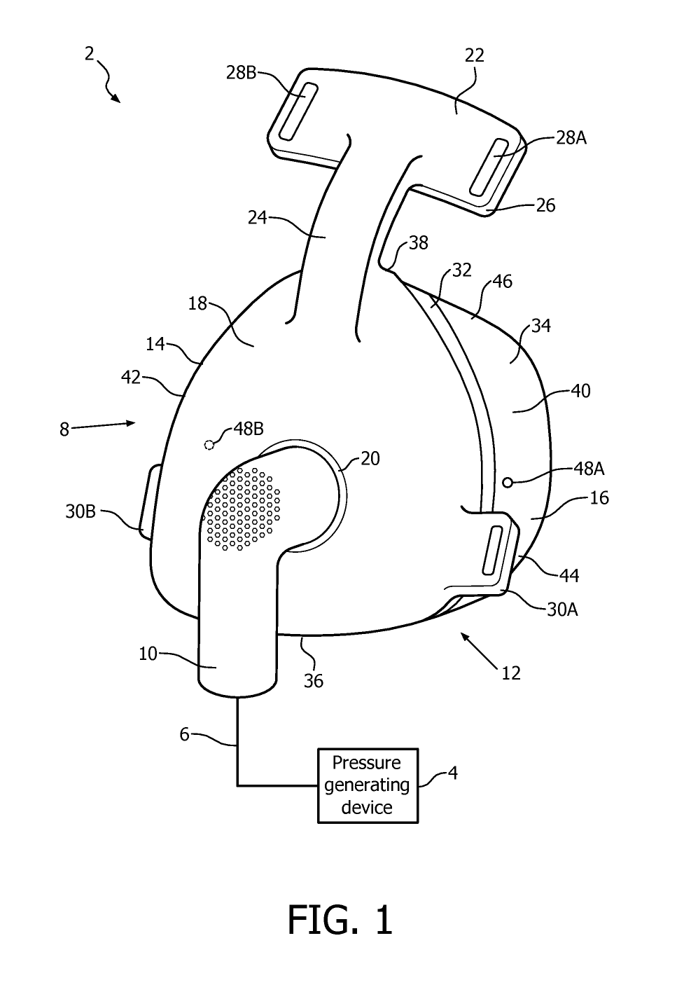

A system 2 adapted to provide a regimen of respiratory therapy to a patient according to one exemplary embodiment is generally shown in FIG. 1. System 2 includes a pressure generating device 4, a delivery conduit 6, and a patient interface device 8 having a fluid coupling conduit 10. Pressure generating device 4 is structured to generate a flow of breathing gas and may include, without limitation, ventilators, constant pressure support devices (such as a continuous positive airway pressure device, or CPAP device), variable pressure devices (e.g., BiPAP®, Bi-Flex®, or C-Flex™ devices manufactured and distributed by Philips Respironics of Murrysville, Pa.), and auto-titration pressure support devices. Delivery conduit 6 is structured to communicate the flow of breathing gas from pressure generating device 4 to patient interface device 8 through fluid coupling conduit 10, which in the illustrated embodiment is an elbow connector. Delivery conduit 6 and patient interface device 8 are often collectively referred to as a patient circuit.

In the exemplary embodiment, patient interface 8 includes a patient sealing assembly 12, which in the illustrated embodiment is a nasal/oral mask. However, other types of patient sealing assemblies, such as, without limitation, a nasal mask, a nasal cushion or a full face mask, which facilitates the delivery of the flow of breathing gas to the airway of a patient, may be substituted for patient sealing assembly 12 while remaining within the scope of the present invention. Patient sealing assembly 12 includes a frame member 14 having a cushion assembly 16 coupled thereto, each of which is described in greater detail below.

In the illustrated embodiment, frame member 14 is made of a rigid or semi-rigid material, such as, without limitation, an injection molded thermoplastic or silicone, and includes a faceplate portion 18 having an opening 20 formed therein. As seen in FIG. 1, fluid coupling conduit 10 is coupled to faceplate portion 18 through opening 20, which configuration allows the flow of breathing gas from pressure generating device 4 to be communicated to an interior space defined by cushion assembly 16, and then to the airway of a patient.

In the exemplary embodiment, frame member 14 also includes a forehead support member 22 that is coupled to faceplate portion 18 by a connecting member 24. As seen in FIG. 1, a forehead cushion 26 is coupled to the rear of forehead support member 22. In the exemplary embodiment, forehead cushion 26 is made of a unitary piece of soft, flexible, cushiony, elastomeric material, such as, without limitation, silicone, an appropriately soft thermoplastic elastomer, a closed cell foam, or any combination of such materials. Forehead support member 22 includes looped connectors 28A, 28B to which the upper straps of a headgear component (not shown) may be attached. In addition, looped connecting elements 30A, 30B are attached to the bottom sides of faceplate portion 18, and are structured to receive and hold the lower straps of a headgear component (not shown).

Cushion assembly 16 in the exemplary embodiment includes a support frame 32 and a sealing cushion 34 coupled to support frame 32. In the illustrated embodiment, support frame 32 is made of a rigid or semi-rigid material, such as, without limitation, an injection molded thermoplastic or silicone, and sealing cushion 34 is defined from a unitary piece of soft, flexible, cushiony, elastomeric material, such as, without limitation, silicone, an appropriately soft thermoplastic elastomer, a closed cell foam, or any combination of such materials. It to be understood that the forehead support structure is optional and can be omitted entirely.

In the illustrated embodiment, patient sealing assembly 12 has a generally triangular shape including a bottom region 36, an apex region 38 located opposite bottom region 36, a first side region 40 and a second side region 42 located opposite first side region 40. As a result, both faceplate portion 18 of frame member 14 and cushion assembly 16 will have associated bottom, apex and first and second side regions (not labeled individually in FIG. 1). In addition, each side region 40, 42 has a respective bottom end 44 and apex end 46 which defines the length of the side region 40, 42.

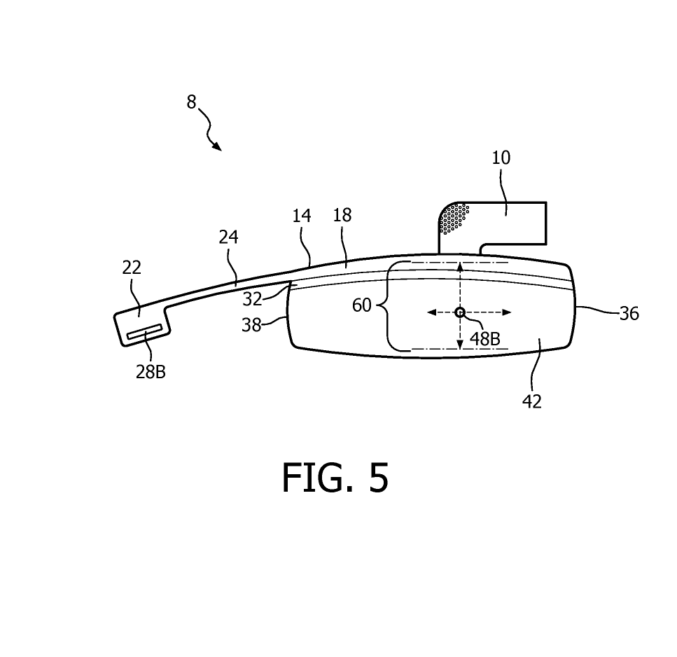

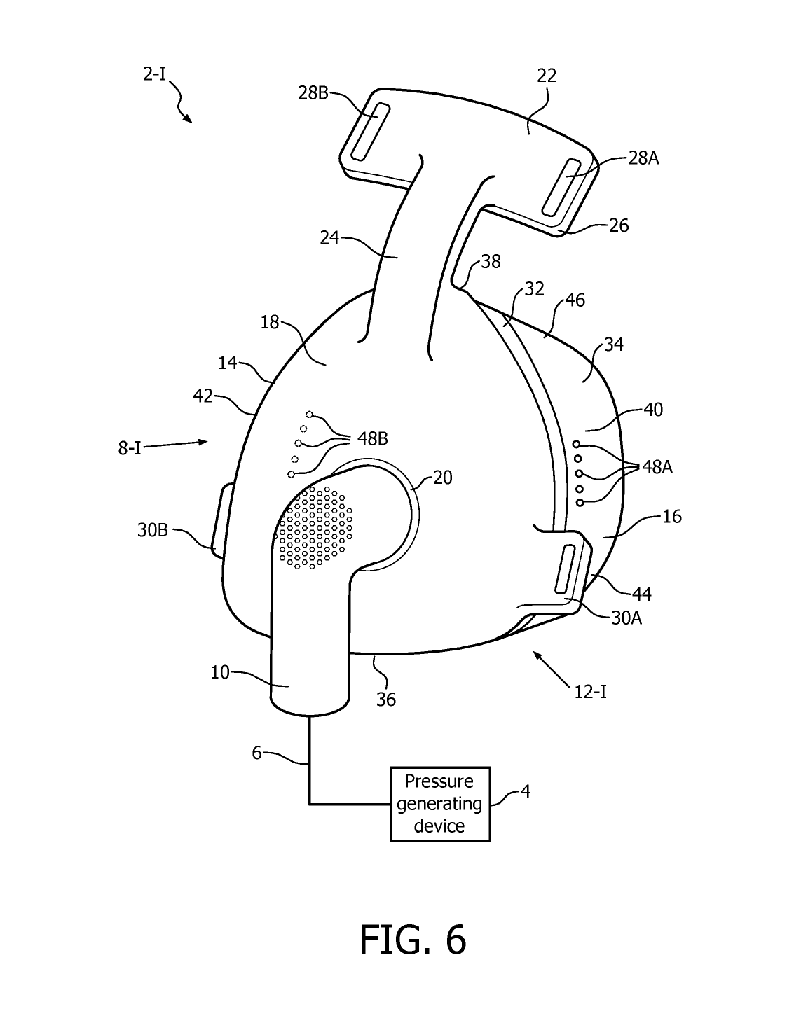

Furthermore, as seen in FIG. 1, an orifice 48A is provided within and extends through side region 40 of patient sealing assembly 12. Similarly, an orifice 48B (shown in phantom lines in FIG. 1) is provided within and extends through side region 42 of patient sealing assembly 12. In the illustrated embodiment, orifices 48A and 48B are provided within and extend through the side regions of sealing cushion 34. It will be appreciated, however, that orifices 48A, 48B may be provided within other portions of the side regions 40, 44, such as in the side regions of faceplate portion 18. See, for example, the embodiments of FIGS. 6-9 described elsewhere herein.

In the exemplary embodiment, orifices 48A and 48B each act as a drain for patient interface device 8 which, when the patient is in various sleeping positions, allow liquid that may condense within patient sealing assembly 12 as described elsewhere herein to freely drain from patient interface device 8, thereby avoiding the undesirable puddling and pooling of such liquids that occurs in the prior art. In addition, in the exemplary embodiment, orifices 48A and 48B each also act as exhalation ports which allow for venting of exhalation gasses.

In one particular exemplary embodiment, orifices 48A, 48B are each placed within particular predefined regions within the side regions 40, 42. As described in greater detail below, the particular predefined regions are selected such that locating orifices 48A and 48B within them will result in one of the orifices 48A, 48B being positioned at the lowest point of patient sealing assembly 12 when the patient is sleeping on one of his or her sides. This positioning will promote/facilitate the draining of the condensed liquid from patient interface device 8.

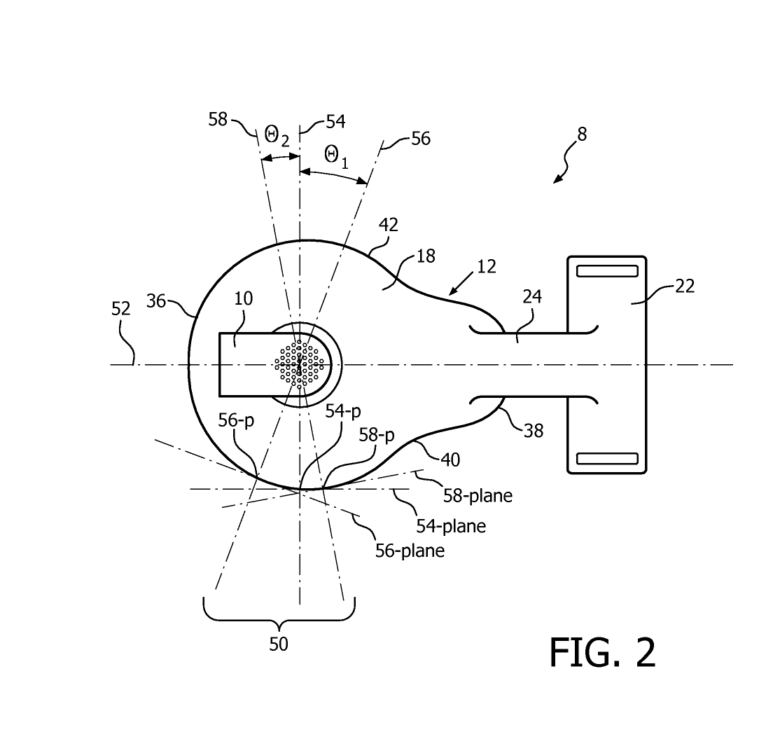

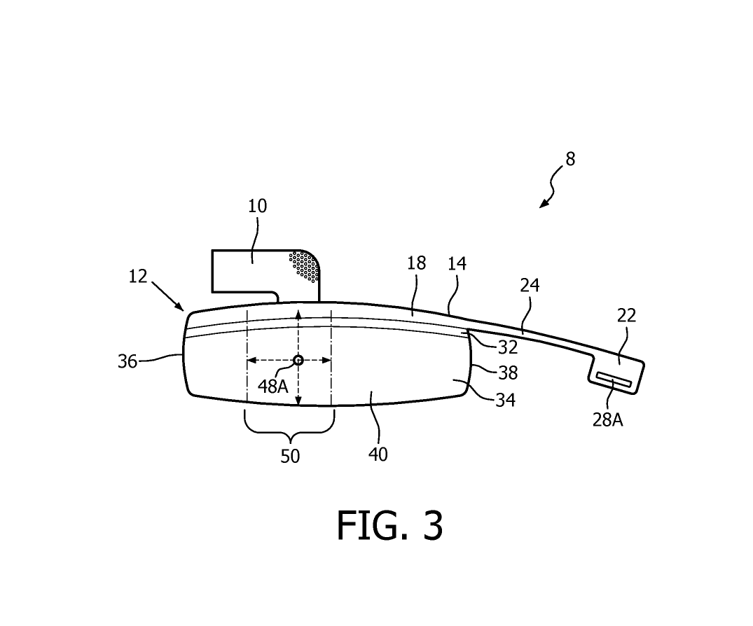

FIG. 2 is a schematic diagram of patient interface device 8 showing one such particular predefined region, labeled with reference numeral 50, according to one particular exemplary embodiment. In FIG. 2, a top plan view of patient interface device 8 is provided in order to illustrate the predefined region 50. FIG. 2 includes a line 52 that bisects patient sealing assembly 12 in a direction extending from a middle of bottom region 36 through a middle of apex region 38. FIG. 2 also includes a first patient position line 54 that intersects and is normal to line 52, a second patient position line 56 offset from the first patient position line 54 in a first direction by angle Θ₁ (line 56 intersects line 52 at the same point as line 54), and a third patient position line 58 offset from the first patient position line 54 in a second, opposite direction by angle Θ₂ (line 58 intersects line 52 at the same point as line 54).

First patient position line 54 represents a plane through patient sealing assembly 12 that will be oriented perfectly vertically when the patient is in a sleeping position wherein his or her head is positioned perfectly horizontally (i.e., such that bisecting line 52 is positioned perfectly horizontally). In that orientation, the lowest (i.e., lower-most) point of patient sealing assembly 12, identified as 54-P, will be defined by the point (or points) at which a plane 54-plane that is normal to first patient position line 54 is tangent to patient sealing assembly 12.

Second patient position line 56 represents a plane through patient sealing assembly 12 that will be oriented perfectly vertically when the patient is in another, alternate sleeping position wherein his or her head is tilted upwardly in a first direction (e.g., upwardly on a pillow) in a manner such that bisecting line 52 has been rotated by an amount equal to the angle Θ₁. In this orientation, the lowest (i.e., lower-most) point of patient sealing assembly 12, identified as 56-P, will be defined by the point (or points) at which a plane 56-plane that is normal to second patient position line 56 is tangent to patient sealing assembly 12.

Third patient position line 58 represents a plane through patient sealing assembly 12 that will be oriented perfectly vertically when the patient is in still another alternate sleeping position wherein his or her head is tilted downwardly in a second direction in a manner such that bisecting line 52 has been rotated by an amount equal to the angle Θ₂. In this orientation, the lowest (i.e., lower-most) point of patient sealing assembly 12, identified as 58-P, will be defined by the point (or points) at which a plane 58-plane that is normal to third patient position line 58 is tangent to patient sealing assembly 12.

In the manner just described, second patient position line 56 and third patient position line 58 thus define patient positioning extremes resulting from head rotation in opposite directions during sleep. The points 56-P and 58-P as described above also define beginning and end points, respectively, of the predefined region 50 of the present embodiment as it extends in a direction along the length of side region 40. This is shown schematically in FIG. 3. As shown by the dotted arrows in FIG. 3, orifice 48A in this exemplary embodiment may be placed anywhere within the predefined region 50 of side region 40, including within faceplate portion 18. By positioning the orifice 48A in side region 40 within the predefined region 50, patient sealing assembly 12 ensures that orifice 48A will be at or close to the then lowest point of patient sealing assembly 12 as patient sealing assembly 12 rotates with patient position changes, thereby facilitating the draining of liquid that has condensed within patient interface device 8. As will be appreciated, in this embodiment, orifice 48B will be positioned in a similarly defined region 50 in side region 42.

In one particular exemplary embodiment, Θ₁ described above is between 15 and 45 degrees, with one specific implementation being 30 degrees. In another particular exemplary embodiment, Θ₂ described above is between 0 and 20 degrees, with one specific implementation being 10 degrees.

In another particular exemplary embodiment, an orifice 48A, 48B is placed at point 54-P on each side region 40, 42. In yet another particular exemplary embodiment, an orifice 48A, 48B is also placed at points 56-P and 58-P on each side region 40, 42.

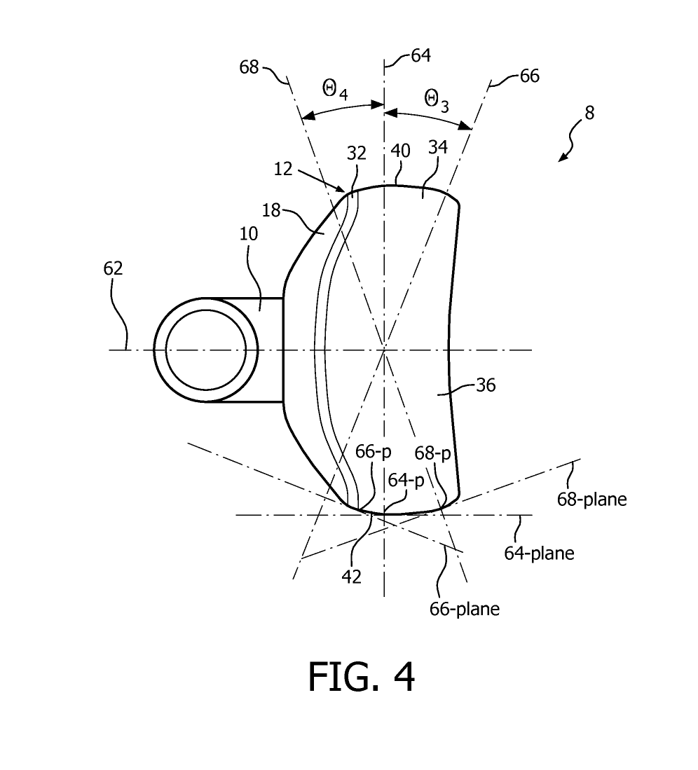

FIG. 4 is a schematic diagram of patient interface device 8 showing another such particular predefined region, labeled with reference numeral 60, according to an alternative particular exemplary embodiment. In FIG. 4, a front elevational view of patient interface device 8 is provided in order to illustrate the predefined region 60. FIG. 4 includes a line 62 that bisects patient sealing assembly 12 in a direction extending from a middle of the top surface of faceplate portion 18 through a middle of a bottom surface of sealing cushion 34. FIG. 4 also includes a fourth patient position line 64 that intersects and is normal to line 62, a fifth patient position line 66 offset from the fourth patient position line 64 in a first direction by angle Θ₃ (line 66 intersects line 62 at the same point as line 64), and a sixth patient position line 68 offset from the fourth patient position line 64 in a second, opposite direction by angle Θ₄ (line 68 intersects line 62 at the same point as line 64).

Fourth patient position line 64 represents a plane through patient sealing assembly 12 that will be oriented perfectly vertically when the patient is in a sleeping position wherein his or her head is positioned perfectly horizontally (i.e., such that bisecting line 62 is positioned perfectly horizontally). In that orientation, the lowest (i.e., lower-most) point of patient sealing assembly 12, identified as 64-P, will be defined by the point (or points) at which a plane 64-plane that is normal to fourth patient position line 64 is tangent to patient sealing assembly 12.

Fifth patient position line 66 represents a plane through patient sealing assembly 12 that will be oriented perfectly vertically when the patient is in another, alternate sleeping position wherein his or her head is tilted downwardly in a first direction (e.g., downwardly with face toward the bed) in a manner such that bisecting line 62 has been rotated by an amount equal to the angle Θ₃. In this orientation, the lowest (i.e., lower-most) point of patient sealing assembly 12, identified as 66-P, will be defined by the point (or points) at which a plane 66-plane that is normal to fifth patient position line 66 is tangent to patient sealing assembly 12.

Sixth patient position line 68 represents a plane through patient sealing assembly 12 that will be oriented perfectly vertically when the patient is in still another alternate sleeping position wherein his or her head is tilted upwardly (e.g., upwardly with face toward the ceiling) in a second direction in a manner such that bisecting line 62 has been rotated by an amount equal to the angle Θ₄. In this orientation, the lowest (i.e., lower-most) point of patient sealing assembly 12, identified as 68-P, will be defined by the point (or points) at which a plane 68-plane that is normal to sixth patient position line 68 is tangent to patient sealing assembly 12.

In the manner just described, fifth patient position line 66 and sixth patient position line 68 thus define patient positioning extremes resulting from head rotation in opposite directions during sleep (as will be appreciated, the direction of head rotation in this embodiment is substantially normal to the direction of head rotation in the embodiment of FIGS. 2 and 3). The points 66-P and 68-P as described above also define beginning and end points, respectively, of the predefined region 60 of the present embodiment as it extends in a direction along the width of side region 42. This is shown schematically in FIG. 5. As shown by the dotted arrows in FIG. 5, orifice 48B in this exemplary embodiment may be placed anywhere within the predefined region 60 of side region 40. By positioning the orifice 48B in side region 42 within the predefined region 60, patient sealing assembly 12 ensures that orifice 48B will be at or close to the then lowest point of patient sealing assembly 12 as patient sealing assembly 12 rotates with patient position changes as describe above, thereby facilitating the draining of liquid that has condensed within patient interface device 8. As will be appreciated, in this embodiment, orifice 48A will be positioned in a similarly defined region 60 in side region 40.

In one particular exemplary embodiment, Θ₃ and Θ₄ described above are between 15 and 45 degrees, with one specific implementation being between 20 and 45 degrees and another specific implementation being 30 degrees.

In another particular exemplary embodiment, an orifice 48A, 48B is placed at point 64-P on each side region 40, 42. In yet another particular exemplary embodiment, an orifice 48A, 48B is also placed at points 66-P and 68-P on each side region 40, 42.

FIG. 6 is a schematic diagram of a system 2-I adapted to provide a regimen of respiratory therapy to a patient according to an alternative exemplary embodiment. System 2-I includes many of the same components as system 2, and like components are labeled with like reference numerals. As seen in FIG. 6, system 2-I includes an alternative patient interface device 8-I having an alternative patient sealing assembly 12-I wherein multiple orifices 48A and 48B are provided in the side region of the sealing cushion 16. In the exemplary embodiment, the multiple orifices 48A and 48B are provided in a predefined region (50 and/or 60) as described herein.

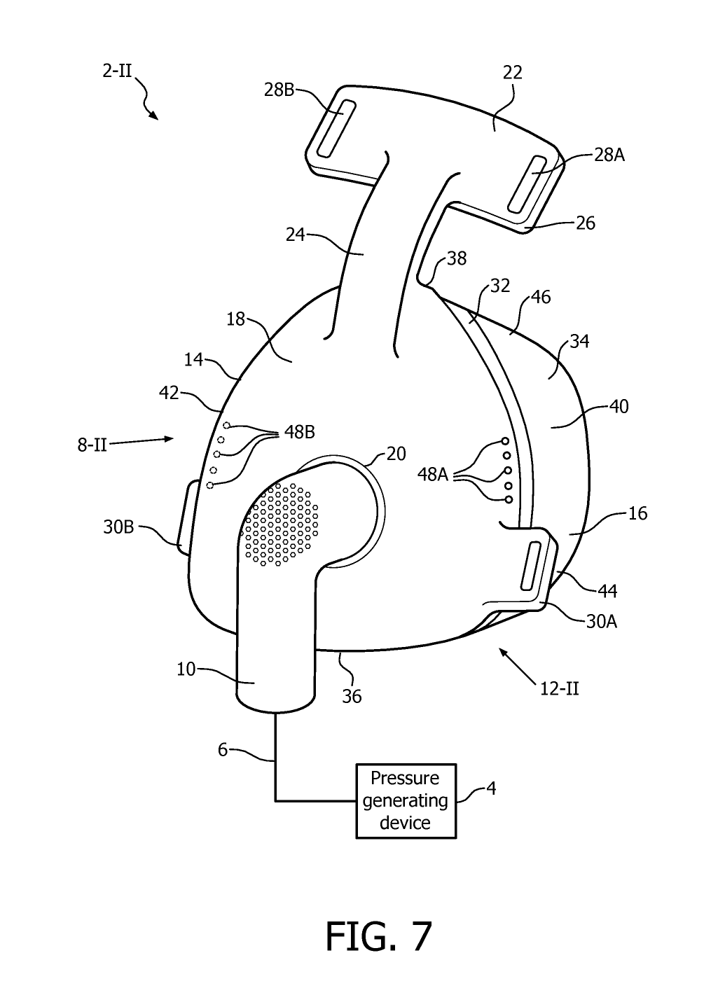

FIG. 7 is a schematic diagram of a system 2-II adapted to provide a regimen of respiratory therapy to a patient according to a further alternative exemplary embodiment. System 2-II includes many of the same components as system 2, and like components are labeled with like reference numerals. As seen in FIG. 7, system 2-II includes an alternative patient interface device 8-II having an alternative patient sealing assembly 12-II wherein multiple orifices 48A and 48B are provided in the side region of the faceplate portion 18. In the exemplary embodiment, the multiple orifices 48A and 48B are provided in a predefined region (50 and/or 60) as described herein.

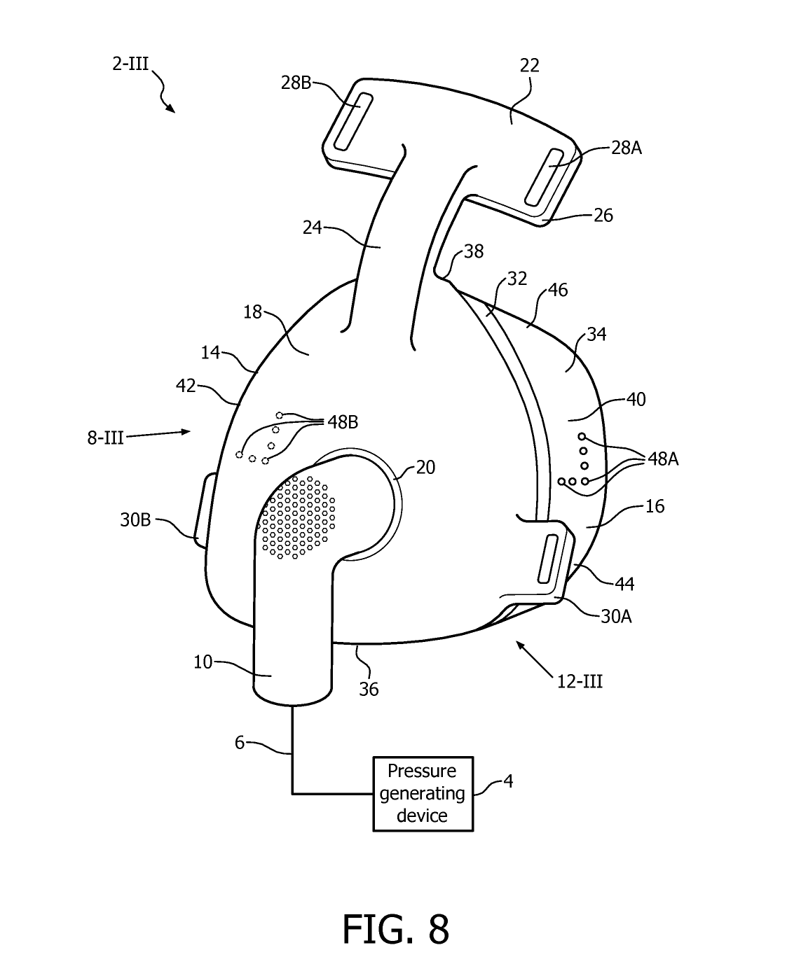

FIG. 8 is a schematic diagram of a system 2-III adapted to provide a regimen of respiratory therapy to a patient according to an alternative exemplary embodiment. System 2-III includes many of the same components as system 2, and like components are labeled with like reference numerals. As seen in FIG. 8, system 2-III includes an alternative patient interface device 8-III having an alternative patient sealing assembly 12-III wherein multiple orifices 48A and 48B are provided in the side region of the sealing cushion 16 in an L-shaped pattern. In the exemplary embodiment, the multiple orifices 48A and 48B are provided in a predefined region (50 and/or 60) as described herein.

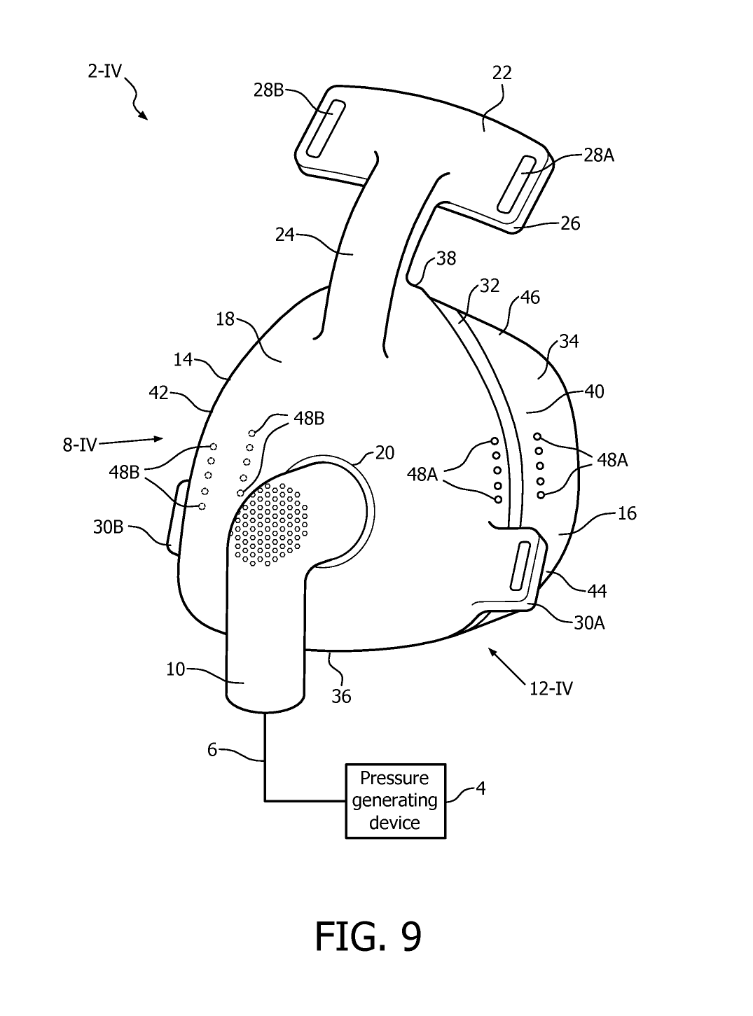

FIG. 9 is a schematic diagram of a system 2-IV adapted to provide a regimen of respiratory therapy to a patient according to an alternative exemplary embodiment. System 2-IV includes many of the same components as system 2, and like components are labeled with like reference numerals. As seen in FIG. 9, system 2-IV includes an alternative patient interface device 8-IV having an alternative patient sealing assembly 12-IV wherein multiple orifices 48A and 48B are provided in the side region of both the faceplate portion 18 and the sealing cushion 16. In the exemplary embodiment, the multiple orifices 48A and 48B are provided in a predefined region (50 and/or 60) as described herein.

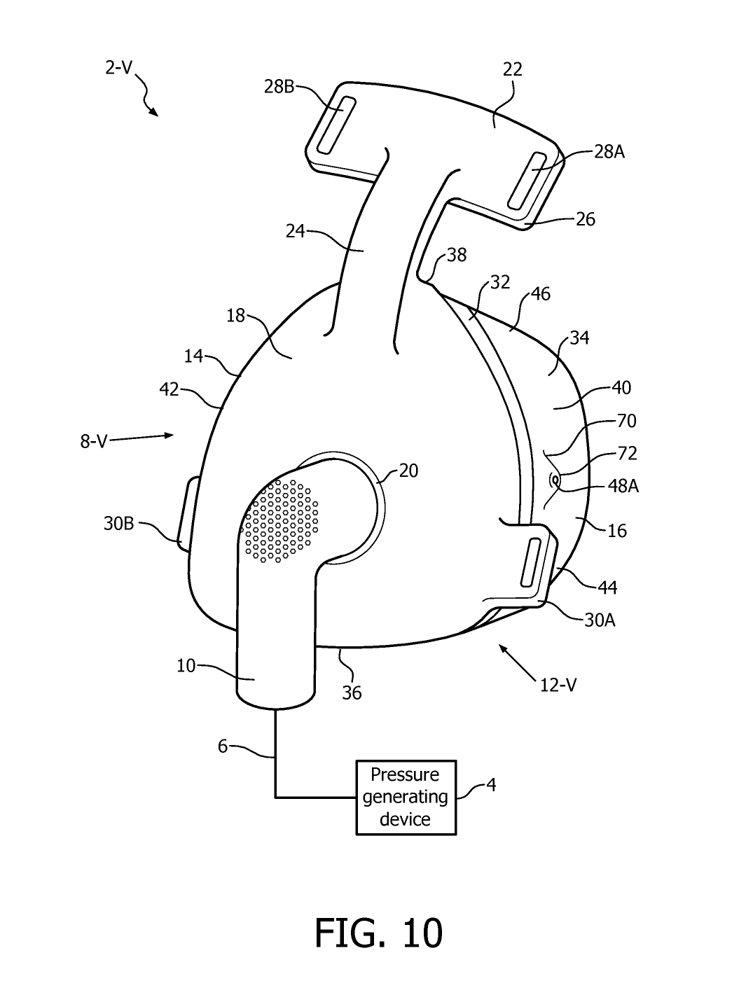

FIG. 10 is a schematic diagram of a system 2-V adapted to provide a regimen of respiratory therapy to a patient according to an alternative exemplary embodiment. System 2-V includes many of the same components as system 2, and like components are labeled with like reference numerals. As seen in FIG. 10, system 2-V includes an alternative patient interface device 8-V having an alternative patient sealing assembly 12-V. In patient sealing assembly 12-V, the side regions 40 and 42 of sealing cushion 16 are each provided with a flow leading geometry in the form of an outwardly extending funnel member 70 (molded as part of sealing cushion 34) having a peak portion 72 in which orifices 48A, 48B are provided. The flow leading geometries lead from an inside of the sealing cushion 34 to an outside of the sealing cushion 34 and encourage condensed liquid to flow toward orifices 48A, 48B from within patient sealing assembly 12-V so that it can be more easily drained from patient interface device 8-V. In the exemplary embodiment, the flow leading geometries are provided in a predefined region (50 and/or 60) as described herein.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A patient interface device, comprising:
a patient sealing assembly structured to engage a face of the patient when the patient interface device is donned by a patient, the patient sealing assembly having a bottom region, a top region opposite the bottom region, and a side region extending from the bottom region to the top region, wherein one or more orifices are provided in the side region, the one or more orifices each extending from an interior of the patient sealing assembly to an exterior of the patient sealing assembly and being structured to allow a liquid that condenses within the patient sealing assembly to drain from the patient sealing assembly;
wherein the patient sealing assembly has a generally triangular shape, wherein the top region comprises an apex region of the patient sealing assembly, wherein the one or more orifices are provided in a predefined region of the side region extending along a length of the side region from a first point of the side region to a second point of the side region, wherein a bisecting line bisects the patient sealing assembly in a direction extending from a middle of the bottom region through a middle of the apex region, wherein a first patient position line is normal to the bisecting line 52, a second patient position line is offset from the first patient position line 54 in a first direction by a first angle having a value from 15 to 45 degrees, and a third patient position line is offset from the first patient position line in a second, opposite direction by a second angle having a value from 0 to 20 degrees, wherein the first patient position line, the second patient position line, and the third patient position line interest the bisecting line at a common point, wherein the first point of the side region is defined by a first location at which a first plane that is normal to the second patient position line is tangent to the patient sealing assembly, and wherein the second point of the side region is defined by a second location at which a second plant that is normal to the third patient position line is tangent to the patient sealing assembly.

2. The patient interface device according to claim 1, wherein the patient sealing assembly includes a frame member having a faceplate portion and a cushion coupled to the frame member.

3. The patient interface device according to claim 2, wherein the one or more orifices are provided in the cushion, the faceplate portion or in both the cushion and the faceplate portion.

4. (canceled)

5. The patient interface device according to claim 3, wherein the one or more orifices comprise a plurality of orifices oriented in an L-shaped pattern.

6.-9. (canceled)

10. The patient interface device according to claim 1, wherein the side region includes a flow leading geometry leading from the interior of the patient sealing assembly to the exterior of the patient sealing assembly, wherein the one or more orifices comprise a first orifice provided in the flow leading geometry, wherein the flow leading geometry is structured to encourage the liquid to flow toward the first orifice from the interior of the patient sealing assembly.

11. The patient interface device according to claim 10, the flow leading geometry comprising an outwardly extending funnel member provided as part of the patient sealing assembly.

12. The patient interface device according to claim 11, wherein the patient sealing assembly includes a frame member having a faceplate portion and a cushion coupled to the frame member, wherein the funnel member is provided as part of the cushion.

13. The patient interface device according to claim 11, wherein the funnel member includes a peak portion, and wherein the first orifice is provided in the peak portion.

14.-16. (canceled)

17. The patient interface device according to claim 1, wherein the first angle is 30 degrees and the second angle is 10 degrees.

18.-19. (canceled)

20. The patient interface device according to claim 1, wherein a first one of the orifices is provided at a third location at which a third plane that is normal to the first patient position line is tangent to the patient sealing assembly, wherein a second one of orifices is provided at the first location and a third one of the orifices is provided at the second location.

21.-22. (canceled)

23. The patient interface device according to claim 2, wherein the one or more orifices are also provided in a second predefined region of the side region extending along a width of the side region from a third point of the side region to a fourth point of the side region, wherein a second bisecting line bisects the patient sealing assembly in a direction extending from a middle of a top surface of the faceplate portion through a middle of a bottom surface of the cushion, wherein a fourth patient position line is normal to the second bisecting line, a fifth patient position line is offset from the fourth patient position line in a first direction by a third angle, and a sixth patient position line is offset from the fourth patient position line in a second, opposite direction by a fourth angle, wherein the fourth patient position line, the fifth patient position line and the sixth patient position line intersect the second bisecting line at a common point, wherein the third point is defined by a third location at which a third plane that is normal to the fifth patient position line is tangent to the patient sealing assembly, and wherein the fourth point is defined by a fourth location at which a fourth plane that is normal to the sixth patient position line is tangent to the patient sealing assembly.

24. The patient interface device according to claim 23, wherein the third angle has a value from 15 to 45 degrees and the fourth angle has a value from 15 to 45 degrees.

25. (canceled)

26. The patient interface device according to claim 24, wherein the third angle and the fourth angle are each 30 degrees.

27. The patient interface device according to claim 23, wherein a first one of the orifices is provided at a fifth location at which a fifth plane that is normal to the first patient position line is tangent to the patient sealing assembly.

28. The patient interface device according to claim 27, wherein a second one of the orifices is provided at the third location and a third one of the orifices is provided at the fourth location.

29. (canceled)

30. A method of providing respiratory therapy to a patient, comprising:
providing a flow of breathing gas to a patient through a patient interface device, the patient interface device including a patient sealing assembly engaging a face of the patient, the patient sealing assembly having a bottom region, a top region opposite the bottom region, and a side region extending from the bottom region to the top region, wherein one or more orifices are provided in the side region, the one or more orifices each extending from an interior of the patient sealing assembly to an exterior of the patient sealing assembly, wherein the patient sealing assembly has a generally triangular shape, wherein the top region comprises an apex region of the patient sealing assembly, wherein the one or more orifices are provided in a predefined region of the side region extending along a length of the side region from a first point of the side region to a second point of the side region, wherein a bisecting line bisects the patient sealing assembly in a direction extending from a middle of the bottom region through a middle of the apex region, wherein a first patient position line is normal to the bisecting line 52, a second patient position line is offset from the first patient position line 54 in a first direction by a first angle having a value from 15 to 45 degrees, and a third patient position line is offset from the first patient position line in a second, opposite direction by a second angle having a value from 0 to 20 degrees, wherein the first patient position line, the second patient position line and the third patient position line intersect the bisecting line at a common point, wherein the first point of the side region is defined by a first location at which a first plane that is normal to the second patient position line is tangent to the patient sealing assembly, and wherein the second point of the side region is defined by a second location at which a second plane that is normal to the third patient position line is tangent to the patient sealing assembly; and
draining a liquid that condenses within the patient sealing assembly during the therapy from the patient sealing assembly through the one or more orifices.
